(19) United States
(12) Patent Application Publication
(10) Pub. No.: US 2010/0261689 A1
(43) Pub. Date: Oct. 14, 2010

(54) PYRROLO [1,2-C] IMIDAZOLE DERIVATIVES FOR USE IN THE PROPHYLAXIS OR TREATMENT OF CANCER WHICH IS REFRACTORY TO KNOWN CANCER THERAPIES

(76) Inventors: Masuo Yamaoka, Tsukuba-Shi (JP); Takahito Hara, Tsukuba-Shi (JP)

Correspondence Address: EDWARDS ANGELL PALMER & DODGE LLP, P.O. BOX 55874, BOSTON, MA, 02205, US

(21) Appl. No.: 12/740,677

(22) PCT Filed: Oct. 28, 2008
(86) PCT No.: PCT/JP2008/069987

§ 371 Date: Apr. 29, 2010

(30) Foreign Application Priority Data
Oct. 29, 2007 (JP) 2007-280813

(51) Int. Cl. A61K 31/567; A61P 35/00; A61K 31/4188

(52) U.S. Cl. 514/171; 514/393

(57) ABSTRACT

The present invention mainly aims to provide a drug for the prophylaxis or treatment of androgen-independent prostate cancer, which is highly useful as a pharmaceutical agent.

The present invention provides a drug for the prophylaxis or treatment of androgen-independent prostate cancer, containing a steroid C_{17,20} lyase inhibitor, particularly, a compound represented by the formula (I):

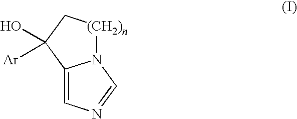

wherein n is an integer of 1 to 3, and Ar is an aromatic ring optionally having substituent(s), or a salt thereof or a prodrug thereof.

### TECHNICAL FIELD

The present invention relates to a drug for the prophylaxis or treatment of androgen-independent prostate cancer.

### BACKGROUND OF THE INVENTION

Prostate cancer is a cancer mainly developed by elderly men, where androgen is deeply involved in the progression thereof. Therefore, inhibition of the production or function of androgen enables suppression of the tumor growth. For the treatment of prostate cancer by the inhibition of the production or function of androgen, surgical castration such as orchiectomy and the like, castration with a gonadotropin-releasing hormone (GnRH) agonist, androgen signal block with an antiandrogen drug, and inhibition of androgen production with an estrogen drug are employed.

As a therapeutic drug for prostate cancer, diethylstylbestrol, chlormadinone acetate, cyproterone acetate, goserelin acetate, buserelin acetate, leuprorelin acetate, ganirelix, flutamide, bicalutamide, nilutamide, dutasteride, finasteride, dexamethasone, prednisolone, ketoconazole, lyase inhibitor and the like are known (e.g., see patent reference 1). Particularly, surgical castration such as orchiectomy and the like, castration with a GnRH agonist and androgen signal block with an antiandrogen drug are highly useful treatment methods since they cause a fewer side effects and show high rates of effect.

When an LHRH (luteinizing hormone-releasing hormone; same as GnRH) agonist is administered to prostate cancer patients, serum testosterone increases temporarily, and the risk of tumor recurrence and aggravation increases. On the other hand, it is known that an increase in serum testosterone is suppressed by a combined used of an LHRH agonist and a steroid biosynthesis inhibitor (aminoglutethimide, ketoconazole) (e.g., see non-patent reference 1).

In the actual site of a cancer treatment, the problem is that the effect of a therapeutic drug is attenuated when patients acquire resistance to the therapeutic drug, and cancer recurrence, metastasis and the like occur. Thus, the development of a pharmaceutical agent for administration to cancer patients who have acquired resistance to therapeutic drugs is demanded. Even in prostate cancer patients who underwent a treatment for inhibition of the production or function of androgen, tumor may acquire growth ability again. Prostate cancer that has acquired growth ability again after once suppressing the growth ability by inhibition of the production or function of androgen by some therapy such as orchiectomy, hormone therapy and the like, is called androgen-independent prostate cancer.

As the mechanism of prostate cancer acquiring the growth ability again, (1) stimulation of tumor growth by androgen at a lower concentration, (2) decreased selectivity of ligand due to mutation of androgen receptor (e.g., see non-patent reference 2), (3) increased expression of enzyme that converts low active androgen (e.g., dehydroepiandrosterone; DHEA, dehydroepiandrosterone sulfate; DHEA-S) produced by adrenal gland, which cannot be suppressed by surgical castration such as orchiectomy and the like, castration with a GnRH agonist or inhibition of androgen production with an estrogen drug, to high active androgen (e.g., testosterone, dihydrotestosterone) (e.g., see non-patent reference 3) and the like are considered.

In addition, the effectiveness of compound CB7630 (abiraterone acetate), which is a 17α-hydroxylase and steroid C_{17,20} lyase inhibitor, for prostate cancer having resistance to anticancer drug docetaxel is being studied in clinical tests (e.g., see non-patent reference 4).

However, a pharmaceutical agent for androgen-independent prostate cancer has not been found yet.

Due to the above-mentioned situation, a pharmaceutical agent that overcomes androgen-independent prostate cancer is desired in actual clinical sites.

patent reference 1: WO2002/40484
non-patent reference 1: The rise in testicular androgens during the first days of treatment with an LHRH agonist in the dog can be blocked by aminoglutethimide or ketoconazole; J. Steroid Biochem. vol. 31, No. 6, pages 963-970 (1988)
non-patent reference 2: Novel mutations of androgen receptor: a possible mechanism of bicalutamide withdrawal syndrome; T Hara et al. Cancer Research vol. 63, pages 149-153 (2003)
non-patent reference 3: Increased expression of genes converting adrenal androgens to testosterone in androgen-independent prostate cancer; M Stanbrough et al. Cancer Research vol. 66, pages 2815-2825 (2006) non-patent reference 4: Cougar Biotechnology news release “Cougar Biotechnology Announces Initiation of Phase III Trial of CB7630 (Abiraterone Acetate)” (Apr. 29, 2008)

### DISCLOSURE OF THE INVENTION

### Problems to be Solved by the Invention

The present invention aims to provide a drug for the prophylaxis or treatment of androgen-independent prostate cancer, which is highly useful as a pharmaceutical agent.

### Means of Solving the Problems

The present inventors have conducted intensive studies in an attempt to find a superior drug for the prophylaxis or treatment of androgen-independent prostate cancer and found that a steroid C_{17,20} lyase inhibitor (particularly, a compound represented by the formula (I):

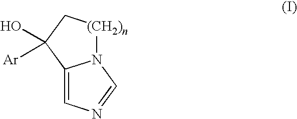

wherein n is an integer of 1 to 3 and Ar is an aromatic ring optionally having substituent(s)) (hereinafter to be referred to as “compound (I)”)) is useful for the prophylaxis or treatment of androgen-independent prostate cancer. In addition, the present inventors have found that a pharmaceutical agent comprising a steroid C_{17,20} lyase inhibitor (particularly, compound (I)) and a concomitant drug in combination is useful for the prophylaxis or treatment of androgen-independent prostate cancer. Moreover, the present inventors have found that a pharmaceutical agent comprising compound (I) and a concomitant drug in combination is useful for administration to cancer patients who developed resistance to a therapeutic drug (anticancer drug). Furthermore, the present inventors have found that a pharmaceutical agent comprising compound (I) and a concomitant drug in combination is useful for the prevention of acquisition of resistance to cancer.

The present invention has been completed based on these findings.

Accordingly, the present invention relates to the following [1]-[36].

[1] A drug for the prophylaxis or treatment of androgen-independent prostate cancer comprising a steroid C_{17,20} lyase inhibitor (hereinafter to be also referred to as “a drug for the prophylaxis or treatment of AIPC of the present invention”).
[2] The drug of the above-mentioned [1], wherein the steroid C_{17,20} lyase inhibitor is compound (I) or a salt thereof or a prodrug thereof.
[3] The drug of the above-mentioned [2], wherein the Ar is a monocyclic or bicyclic aromatic fused ring optionally having substituent(s).
[4] The drug of the above-mentioned [2], wherein the Ar is an aromatic ring having 5 to 10 atoms containing 0 to 4 hetero atoms as ring-constituting atom(s), which ring is optionally substituted and is bonded at carbon atom.
[5] The drug of the above-mentioned [2], wherein the Ar is a group represented by the formula:

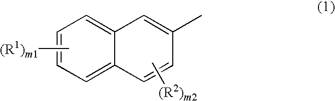

wherein m1 is an integer of 1 to 4, m2 is an integer of 0 to 3, and R¹ and R² are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s), a group represented by the formula:

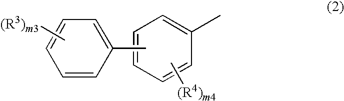

wherein m3 is an integer of 1 to 5, m4 is an integer of 0 to 4, R³ and R⁴ are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s), or a group represented by the formula:

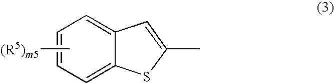

wherein m5 is an integer of 1 to 4, R⁵ is a hydrogen atom, a hydroxyl group optionally having substituent(s), a thiol group optionally having substituent(s), an amino group optionally having substituent(s), an acyl group, a halogen atom or a hydrocarbon group optionally having substituent(s).
[6] The drug of the above-mentioned [2], wherein the Ar is a group represented by the formula:

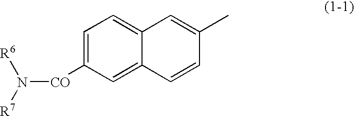

wherein R⁶ and R⁷ are the same or different and each independently is a hydrogen atom or a lower alkyl group, or a group represented by the formula:

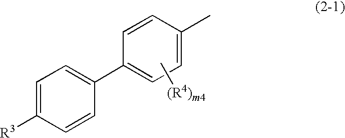

wherein m4 is an integer of 0 to 4, and R³ and R⁴ are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s).
[7] The drug of the above-mentioned [2], wherein the Ar is a group represented by the formula:

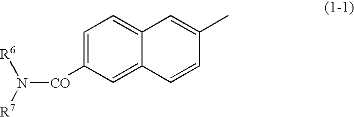

wherein R⁶ and R⁷ are the same or different and each independently is a hydrogen atom or a lower alkyl group.
[8] The drug of the above-mentioned [2], wherein compound (I) is an enantiomer wherein the steric configuration of hydrocarbon bonded to a hydroxyl group is an S configuration.
[9] The drug of the above-mentioned [2], wherein compound (I) is an enantiomer wherein the steric configuration of hydrocarbon bonded to a hydroxyl group is an R configuration.
[10] The drug of the above-mentioned [2], wherein the compound (I) is selected from the group consisting of the following compounds:
(±)-7-(5-methoxybenzo[b]thiophen-2-yl)-6,7-dihydro-5H-pyrrolo[1,2-c]imidazol-7-ol,
(±)-7-(5-fluorobenzo[b]thiophen-2-yl)-6,7-dihydro-5H-pyrrolo[1,2-c]imidazol-7-ol,
(±)-7-(4′-fluoro[1,1′-biphenyl]-3-yl)-6,7-dihydro-5H-pyrrolo[1,2-c]imidazol-7-ol,
(±)-7-(4′-fluoro[1,1′-biphenyl]-4-yl)-6,7-dihydro-5H-pyrrolo[1,2-c]imidazol-7-ol,
(±)-6-(7-hydroxy-6,7-dihydro-5H-pyrrolo[1,2-c]imidazol-7-yl)-N-methyl-2-naphthamide,
(±)-N-ethyl-6-(7-hydroxy-6,7-dihydro-5H-pyrrolo[1,2-c]imidazol-7-yl)-2-naphthamide,
(±)-6-(7-hydroxy-6,7-dihydro-5H-pyrrolo[1,2-c]imidazol-7-yl)-N-isopropyl-2-naphthamide, and
(±)-6-(7-hydroxy-6,7-dihydro-5H-pyrrolo[1,2-c]imidazol-7-yl)-2-naphthamide.
   [11] The drug of the above-mentioned [2], wherein the compound (I) is selected from the group consisting of the following compounds:
(±)-7-(4′-fluoro[1,1′-biphenyl]-3-yl)-6,7-dihydro-5H-pyrrolo[1,2-c]imidazol-7-ol,
(±)-7-(4′-fluoro[1,1′-biphenyl]-4-yl)-6,7-dihydro-5H-pyrrolo[1,2-c]imidazol-7-ol,
(±)-6-(7-hydroxy-6,7-dihydro-5H-pyrrolo[1,2-c]imidazol-7-yl)-N-methyl-2-naphthamide, and
(±)-6-(7-hydroxy-6,7-dihydro-5H-pyrrolo[1,2-c]imidazol-7-yl)-2-naphthamide.
   [12] A drug for the prophylaxis or treatment of androgen-independent prostate cancer comprising (+)-7-(4′-fluoro[1,1′-biphenyl]-3-yl)-6,7-dihydro-5H-pyrrolo[1,2-c]imidazol-7-ol or a salt thereof.
   [13] A drug for the prophylaxis or treatment of androgen-independent prostate cancer comprising (−)-7-(4′-fluoro[1,1′-biphenyl]-3-yl)-6,7-dihydro-5H-pyrrolo[1,2-c]imidazol-7-ol or a salt thereof.
   [14] A drug for the prophylaxis or treatment of androgen-independent prostate cancer comprising (+)-7-(4′-fluoro[1,1′-biphenyl]-4-yl)-6,7-dihydro-5H-pyrrolo[1,2-c]imidazol-7-ol or a salt thereof.
   [15] A drug for the prophylaxis or treatment of androgen-independent prostate cancer comprising (−)-7-(4′-fluoro[1,1′-biphenyl]-4-yl)-6,7-dihydro-5H-pyrrolo[1,2-c]imidazol-7-ol or a salt thereof.
   [16] A drug for the prophylaxis or treatment of androgen-independent prostate cancer comprising (+)-6-(7-hydroxy-6,7-dihydro-5H-pyrrolo[1,2-c]imidazol-7-yl)-N-methyl-2-naphthamide or a salt thereof.
   [17] A drug for the prophylaxis or treatment of androgen-independent prostate cancer comprising (−)-6-(7-hydroxy-6,7-dihydro-5H-pyrrolo[1,2-c]imidazol-7-yl)-N-methyl-2-naphthamide or a salt thereof.
   [18] A drug for the prophylaxis or treatment of androgen-independent prostate cancer comprising (+)-6-(7-hydroxy-6,7-dihydro-5H-pyrrolo[1,2-c]imidazol-7-yl)-2-naphthamide or a salt thereof.
   [19] A drug for the prophylaxis or treatment of androgen-independent prostate cancer comprising (−)-6-(7-hydroxy-6,7-dihydro-5H-pyrrolo[1,2-c]imidazol-7-yl)-2-naphthamide or a salt thereof.
   [20] The drug of the above-mentioned [1], which is used in combination with a concomitant drug.
   [21] The drug of the above-mentioned [20], wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof.
   [22] The drug of the above-mentioned [20], wherein the concomitant drug is a GnRH receptor agonist or a GnRH receptor antagonist.
   [23] A therapeutic drug for cancer having resistance to an anticancer drug, which comprises compound (I) or a salt thereof or a prodrug thereof, and a concomitant drug in combination (hereinafter to be also referred to as “a therapeutic drug for cancer having resistance to an anticancer drug of the present invention”).
   [24] The drug of the above-mentioned [23], wherein the anticancer drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof.
   [25] The drug of the above-mentioned [23], wherein the anticancer drug is a GnRH receptor agonist or a GnRH receptor antagonist.
   [26] The drug of the above-mentioned [23], wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof.
   [27] The drug of the above-mentioned [23], wherein the concomitant drug is a GnRH receptor agonist or a GnRH receptor antagonist.
   [28] A drug for preventing acquisition of resistance of cancer to an anticancer drug, which comprises compound (I) or a salt thereof or a prodrug thereof, and a concomitant drug in combination (hereinafter to be also referred to as “a drug for preventing acquisition of resistance of cancer to an anticancer drug of the present invention”).
   [29] The drug of the above-mentioned [28], wherein the anticancer drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof.
   [30] The drug of the above-mentioned [28], wherein the anticancer drug is a GnRH receptor agonist or a GnRH receptor antagonist.
   [31] The drug of the above-mentioned [28], wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof.
   [32] The drug of the above-mentioned [28], wherein the concomitant drug is a GnRH receptor agonist or a GnRH receptor antagonist.
   [33] A method for the prophylaxis or treatment of androgen-independent prostate cancer in a mammal, which comprises administering an effective amount of a steroid C_{17,20} lyase inhibitor, or effective amounts of a steroid C_{17,20} lyase inhibitor and a concomitant drug to the mammal.
   [34] A method of treating cancer having resistance to an anticancer drug, or preventing acquisition of resistance of cancer to an anticancer drug, which comprises administering effective amounts of compound (I) or a salt thereof or a prodrug thereof, and a concomitant drug to a mammal.
   [35] Use of a steroid C_{17,20} lyase inhibitor, or a steroid C_{17,20} lyase inhibitor and a concomitant drug for the production of a drug for the prophylaxis or treatment of androgen-independent prostate cancer.
   [36] Use of compound (I) or a salt thereof or a prodrug thereof, and a concomitant drug for the production of a therapeutic drug for cancer having resistance to an anticancer drug, or a drug for preventing acquisition of resistance of cancer to an anticancer drug.

### EFFECT OF THE INVENTION

The drug for the prophylaxis or treatment of AIPC of the present invention is useful since it can be administered to patients with androgen-independent prostate cancer, posing problems in actual clinical sites. In addition, the therapeutic drug for cancer having resistance to an anticancer drug of the present invention is useful for administration to cancer patients who acquired resistance to an anticancer drug. Moreover, the drug for preventing acquisition of resistance of cancer to an anticancer drug of the present invention is useful since it can be administered to patients for prevention of cancer recurrence.

### BRIEF DESCRIPTION OF THE DRAWINGS

FIG. 1 shows the serum DHEA concentration of a castrated male cynomolgus administered with a test compound, wherein a black circle (--) shows a test compound (7.5 mg/kg/dose) administration group, and a white circle (-◯-) shows a test compound (15 mg/kg/dose) administration group.

FIG. 2 shows the serum DHEA concentration of a castrated male cynomolgus administered with a vehicle (0.5% methylcellulose) about one month after administration of a test compound, wherein a black circle (--) shows a test compound (7.5 mg/kg/dose) administration group, and a white circle (-◯-) shows a test compound (15 mg/kg/dose) administration group.

FIG. 3 shows the serum testosterone concentration of a castrated male cynomolgus administered with a test compound, wherein a black circle (--) shows a test compound (7.5 mg/kg/dose) administration group, and a white circle (-◯-) shows a test compound (15 mg/kg/dose) administration group.

FIG. 4 shows the serum testosterone concentration of a castrated male cynomolgus administered with a vehicle (0.5% methylcellulose) about one month after administration of a test compound, wherein a black circle (--) shows a test compound (7.5 mg/kg/dose) administration group, and a white circle (-◯-) shows a test compound (15 mg/kg/dose) administration group.

The present invention relates to a drug for the prophylaxis or treatment of androgen-independent prostate cancer, which comprises a steroid C_{17,20} lyase inhibitor.

In the present invention, the “androgen-independent prostate cancer” means a “prostate cancer that has acquired growth ability again after once suppressing the growth ability of tumor by inhibition of the production or function of androgen by some therapy such as orchiectomy, hormone therapy and the like”. In addition, “suppression of growth ability” means a state where decreased blood PSA (Prostate Specific Antigen) concentration, suppression of tumor growth by CT (Computed Tomography), MRI (Magnetic Resonance Imaging), ultrasonication and the like, or alleviation of bone pain is observed in prostate cancer patients who underwent a therapy for inhibiting the production or function of androgen by some treatment such as orchiectomy, hormone therapy and the like. The decreased blood PSA level means that, for example, the blood PSA level becomes 50% or lower than that before treatment.

Moreover, “acquisition of growth ability again” means a state where continuous increase of blood PSA level, tumor growth by a method such as CT, MRI, ultrasonication and the like, or expression or aggravation of bone pain, or new metastatic focus is observed in prostate cancer patients after once suppressing the growth ability of tumor by a therapy for inhibiting the production or function of androgen. The continuous increase of blood PSA level means a state where, for example, an increase of the blood PSA level is observed two or more times successively by a periodic check up.

A steroid C_{17,20} lyase inhibitor can be a compound or composition having a steroid C_{17,20} lyase inhibitory activity and, for example, compound (I) or a salt thereof or a prodrug thereof can be specifically mentioned.

In the present specification, the definition of each symbol in the formulas relating to compound (I) and specific examples of preferable compound (I) are as follows.

n is an integer of 1 to 3, and preferably 1.

m1 is an integer of 1 to 4, preferably 1 or 2, and particularly preferably 1.

m2 is an integer of 0 to 3, preferably 0 or 1, and particularly preferably 0.

m3 is an integer of 1 to 5, preferably 1 to 3, and particularly preferably 1.

m4 is an integer of 0 to 4, preferably 0 or 1, and particularly preferably 0.

m5 is an integer of 1 to 4, preferably 1 or 2, and particularly preferably 1.

Examples of the hydroxyl group optionally having substituent(s) for R¹, R², R³, R⁴ or R⁵ include an unsubstituted hydroxyl group, as well as lower alkoxy (e.g., C₁₋₄ alkoxy group such as methoxy, ethoxy, propoxy etc.), lower alkanoyloxy (e.g., C₁₋₄ alkanoyloxy such as acetyloxy, propionyloxy etc.), carbamoyloxy optionally having substituent(s) (e.g., unsubstituted carbamoyloxy, as well as carbamoyloxy substituted by 1 or 2 C₁₋₄ alkyl groups such as methylcarbamoyloxy, ethylcarbamoyloxy, dimethylcarbamoyloxy, diethylcarbamoyloxy, ethylmethylcarbamoyloxy etc.) and the like.

Examples of the thiol group optionally having substituent(s) for R¹, R², R³, R⁴ or R⁵ include an unsubstituted thiol group, as well as lower alkylthio (e.g., C₁₋₄ alkylthio group such as methylthio, ethylthio, propylthio etc.), lower alkanoylthio (e.g., C₁₋₄ alkanoylthio such as acetylthio, propionylthio etc.) and the like.

Examples of the amino group optionally having substituent(s) for R¹, R², R³, R⁴ or R⁵ include an unsubstituted amino group, as well as lower alkylamino (e.g., C₁₋₄ alkylamino group such as methylamino, ethylamino, propylamino etc.), di-lower alkylamino (e.g., alkylamino such as dimethylamino, diethylamino etc.), C₁₋₄ alkanoylamino (e.g., acetylamino, propionylamino etc.) and the like.

Examples of the acyl group for R¹, R², R³, R⁴ or R⁵ include an alkanoyl group (e.g., C₁₋₆ alkanoyl such as formyl, acetyl, propionyl and the like), an alkylsulfonyl group (e.g., C₁₋₄ alkylsulfonyl such as methylsulfonyl, ethylsulfonyl and the like), an aroyl group (e.g., C₆₋₁₀ aroyl group such as benzoyl, toluoyl, naphthoyl and the like), a carbamoyl group optionally having substituent(s) (e.g., methylcarbamoyl, ethylcarbamoyl, isopropylcarbamoyl), a mono- or di-C₁₋₁₀ alkyl-carbamoyl group such as dimethylcarbamoyl, diethylcarbamoyl and the like, a mono- or di-C₃₋₈ cycloalkyl-carbamoyl group such as cyclopropylcarbamoyl, cyclobutylcarbamoyl and the like, a mono- or di-C₆₋₁₄ aryl-carbamoyl group such as phenylcarbamoyl, diphenylcarbamoyl and the like, a mono- or di-C₇₋₁₆ aralkyl-carbamoyl group such as benzylcarbamoyl, di-benzylcarbamoyl and the like etc.), a sulfamoyl group optionally having substituent(s) (e.g., mono- or di-C₁₋₁₀ alkylsulfamoyl group such as methylsulfamoyl, ethylsulfamoyl, dimethylsulfamoyl, diethylsulfamoyl and the like, mono- or di-C₃₋₈ cycloalkylsulfamoyl group such as cyclopropylsulfamoyl, cyclobutylsulfamoyl and the like, mono- or di-C₆₋₁₄ arylsulfamoyl group such as phenylsulfamoyl, diphenylsulfamoyl and the like, mono- or di-C₇₋₁₆ aralkylsulfamoyl group such as benzylsulfamoyl, di-benzylsulfamoyl and the like etc.), and the like.

Examples of the halogen for R¹, R², R³, R⁴ or R⁵ include fluorine, chlorine, bromine and iodine.

Examples of the “hydrocarbon group” of the “hydrocarbon group optionally having substituent(s)” for R¹, R², R³, R⁴ or R⁵ include a chain hydrocarbon group, a cyclic hydrocarbon group and the like.

Examples of the chain hydrocarbon group include a linear or branched chain hydrocarbon group having a carbon number of 1 to 10, and the like, and specific examples thereof include an alkyl group, an alkenyl group and the like. Among these, an alkyl group is particularly preferable. Examples of the “alkyl group” include a C₁₋₁₀ alkyl group such as methyl, ethyl, n-propyl, isopropyl, butyl, isobutyl, sec-butyl, tert-butyl, n-pentyl, isopentyl, neopentyl, n-hexyl, isohexyl etc., and the like, with preference given to a C₁₋₆ alkyl group (e.g., methyl, ethyl, n-propyl, isopropyl, n-butyl, sec-butyl, tert-butyl etc.). Examples of the “alkenyl group” include a C₂₋₁₀ alkenyl group such as vinyl, 1-propenyl, allyl, isopropenyl, 1-butenyl, 2-butenyl, 3-butenyl, isobutenyl, sec-butenyl etc., and the like, with preference given to a C₂₋₆ alkenyl group (e.g., vinyl, 1-propenyl, allyl etc.). Examples of the “alkynyl group” include a C₂₋₁₀ alkynyl group such as ethynyl, 1-propynyl, propargyl etc., and the like, with preference given to a C₂₋₆ alkynyl group (e.g., ethynyl etc.).

Examples of the cyclic hydrocarbon group include a cyclic hydrocarbon group having a carbon number of 3 to 18, specifically, for example, an alicyclic hydrocarbon group, an aromatic hydrocarbon group and the like.

Examples of the “alicyclic hydrocarbon group” include a monocyclic or condensed polycyclic group having 3 to 10 carbon atoms, specifically a cycloalkyl group, a cycloalkenyl group, a bi- or tricyclic fused ring of these and a C₆₋₁₄ aryl group (e.g., benzene etc.) etc., and the like. Examples of the “cycloalkyl group” include a C₃₋₆ cycloalkyl group such as cyclopropyl, cyclobutyl, cyclopentyl, cyclohexyl etc., and the like, and examples of the “cycloalkenyl group” include a C₃₋₆ cycloalkenyl group such as cyclopropenyl, cyclobutenyl, cyclopentenyl, cyclohexenyl etc., and the like.

Examples of the “aromatic hydrocarbon group” include a monocyclic aromatic hydrocarbon group having 6 to 18 carbon atoms, a condensed polycyclic aromatic hydrocarbon group having 6 to 18 carbon atoms and the like, and specifically, a C₆₋₁₄ aryl group such as phenyl, 1-naphthyl, 2-naphthyl, 2-indenyl, 2-anthryl and the like can be mentioned, with preference given to a C₆₋₁₀ aryl group (e.g., phenyl etc.) and the like.

While the substituent of the “chain hydrocarbon group” in the “hydrocarbon group optionally having substituent(s)” is not particularly limited, for example, halogen atom, hydroxyl group, alkoxy group, acyloxy group, alkylthio group, acylamino group, carboxyl group, alkoxycarbonyl group, oxo group, alkylcarbonyl group, cycloalkyl group, aryl group, aromatic heterocyclic group and the like can be mentioned. These substituents are substituted on a “chain hydrocarbon group” within the chemically acceptable range, where the number of the substituents is 1 to 5, preferably 1 to 3. When the number of the substituents is 2 or more, they may be the same or different.

While the substituent of the “cyclic hydrocarbon group” in the “hydrocarbon group optionally having substituent(s)” is not particularly limited, for example, halogen atom, hydroxyl group, alkoxy group, acyloxy group, alkylthio group, alkylsulfonyl group, mono- or di-alkylamino group, acylamino group, carboxyl group, alkoxycarbonyl group, alkynylcarbonyl group, alkyl group, cycloalkyl group, aryl group, aromatic heterocyclic group and the like can be mentioned. These substituents are substituted on a “cyclic hydrocarbon group” within the chemically acceptable range, where the number of the substituents is 1 to 5, preferably 1 to 3. When the number of the substituents is 2 or more, they may be the same or different.

Examples of the “halogen atom” include fluorine, chlorine, bromine, iodine and the like. Examples of the “alkoxy group” include a C₁₋₁₀ alkoxy group such as methoxy, ethoxy, propoxy, isopropoxy, butoxy, isobutoxy, sec-butoxy, pentyloxy, hexyloxy etc., and the like. Examples of the “acyloxy group” include formyloxy, C₁₋₁₀ alkyl-carbonyloxy (e.g., acetoxy, propionyloxy etc.) and the like. Examples of the “alkylthio group” include a C₁₋₁₀ alkylthio group such as methylthio, ethylthio, propylthio, isopropylthio etc., and the like. Examples of the “alkylsulfonyl group” include a C₁₋₁₀ alkylsulfonyl group such as methylsulfonyl, ethylsulfonyl, propylsulfonyl etc., and the like. Examples of the “acylamino group” include formylamino, di-formylamino, mono- or di-C₁₋₁₀ alkyl-carbonylamino (e.g., acetylamino, propionylamino, butyrylamino, diacetylamino etc.) and the like. Examples of the “mono- or di-alkylamino group” are those similar to the aforementioned lower alkylamino and di-lower alkylamino. Examples of the “alkoxycarbonyl group” include a C₁₋₁₀ alkoxy-carbonyl group such as methoxycarbonyl, ethoxycarbonyl, propoxycarbonyl, isopropoxycarbonyl, butoxycarbonyl etc., and the like. Examples of the “alkylcarbonyl group” include a C₁₋₁₀ alkyl-carbonyl group such as acetyl, propionyl, butyryl, valeryl etc., and the like. Examples of the “alkynylcarbonyl group” include a C₂₋₁₀ alkynyl-carbonyl group such as ethynylcarbonyl, 1-propynylcarbonyl, 2-propynylcarbonyl etc., and the like. Examples of the “cycloalkyl group” include a C₃₋₁₀ cycloalkyl group such as cyclopropyl, cyclobutyl, cyclopentyl, cyclohexyl etc., and the like. Examples of the “aryl group” include a C₆₋₁₄ aryl group such as phenyl, 1-naphthyl, 2-naphthyl etc., and the like. Examples of the “aromatic heterocyclic group” include a mono- to tri-cyclic aromatic heterocyclic group containing, besides carbon atom, one or two kinds of preferably 1 to 4 hetero atoms selected from nitrogen, oxygen and sulfur, and the like. Specifically, for example, thienyl, pyridyl, furylpyrazinyl, pyrimidinyl, imidazolyl, pyrazolyl, thiazolyl, isothiazolyl, oxazolyl, isoxazolyl, pyridazinyl, tetrazolyl, quinolyl, indolyl, isoindolyl and the like can be mentioned. Examples of the “alkyl group” include a C₁₋₁₀ alkyl group such as methyl, ethyl, propyl, isopropyl, butyl, sec-butyl, tert-butyl, pentyl etc., and the like.

The substituent that the aforementioned “hydrocarbon group” optionally has may further have 1 to 5, preferably 1 to 3, substituent(s) as shown below, within a chemically acceptable range. Examples of such substituent include a halogen atom (e.g., fluorine, chlorine, bromine etc.), hydroxyl group and a C₁₋₆ alkoxy group (e.g., methoxy, ethoxy, propoxy, isopropoxy etc.).

Examples of the lower alkyl group for R⁶ or R⁷ include a linear, branched or cyclic alkyl group having a carbon number of 1 to 4. Specifically, for example, methyl, ethyl, n-propyl, isopropyl, butyl, isobutyl, sec-butyl, tert-butyl, cyclopropyl, cyclobutyl and the like can be mentioned.

Examples of the aromatic ring optionally having substituent(s) for Ar include a monocyclic or bicyclic aromatic fused ring optionally having one or more substituent(s) and the like. In addition, an aromatic ring having 5 to 10 atoms containing 0 to 4 hetero atoms as ring-constituting atom(s), which ring is optionally substituted and is bonded at carbon atom (here the aromatic ring is bonded to a condensed imidazole ring in the formula (I) at the carbon atom rather than hetero atom), is also preferably used as Ar.

Examples of the substituent of the aromatic ring optionally having substituent(s) for Ar include a hydroxyl group optionally having substituent(s), a thiol group optionally having substituent(s), an amino group optionally having substituent(s), an acyl group, a halogen atom and a hydrocarbon group optionally having substituent(s). Examples of the “hydroxyl group optionally having substituent(s)”, the “amino group optionally having substituent(s)”, the “acyl group”, the “halogen atom” and the “hydrocarbon group optionally having substituent(s)” include those exemplified for the above-mentioned R¹, R², R³, R⁴ and R⁵.

In the formula (I), Ar is a group represented by the aforementioned formula (1), (2) or (3). Particularly, in the formula (I), Ar is preferably a group represented by the formula (1) or the formula (2), and particularly preferably a group represented by the formula (1). Among the groups represented by the formula (1), a group represented by the aforementioned formula (1-1) is more preferable, and among the groups represented by the formula (1-1), a group wherein both R⁶ and R⁷ are hydrogen atoms, or one of them is a hydrogen atom and the other is a methyl group or an ethyl group, is particularly preferable.

Among the groups represented by the formula (2), a group represented by the aforementioned formula (2-1) is more preferable, and among the groups represented by the formula (2-1), a group wherein m4 is 0 and R³ is a halogen atom is particularly preferable.

Compound (I) has one or more asymmetric carbons in one molecule. Both R configuration and S configuration due to these asymmetric carbons are encompassed in compound (I), and as such compound, a compound wherein the steric configuration (absolute configuration) of the carbon atom bonded to a hydroxyl group (i.e., carbon atom shown by * in the formula:

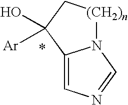

wherein each symbol is as defined above) is S configuration is preferable.

Preferable specific examples of compound (I) include the following compounds.
(±)-7-(5-methoxybenzo[b]thiophen-2-yl)-6,7-dihydro-5H-pyrrolo[1,2-c]imidazol-7-ol,
(±)-7-(5-fluorobenzo[b]thiophen-2-yl)-6,7-dihydro-5H-pyrrolo[1,2-c]imidazol-7-ol,
(±)-7-(4′-fluoro[1,1′-biphenyl]-3-yl)-6,7-dihydro-5H-pyrrolo[1,2-c]imidazol-7-ol,
(±)-7-(4′-fluoro[1,1′-biphenyl]-4-yl)-6,7-dihydro-5H-pyrrolo[1,2-c]imidazol-7-ol,
(±)-6-(7-hydroxy-6,7-dihydro-5H-pyrrolo[1,2-c]imidazol-7-yl)-N-methyl-2-naphthamide,
(±)-N-cyclopropyl-6-(7-hydroxy-6,7-dihydro-5H-pyrrolo[1,2-c]imidazol-7-yl)-2-naphthamide,
(±)-N-ethyl-6-(7-hydroxy-6,7-dihydro-5H-pyrrolo[1,2-c]imidazol-7-yl)-2-naphthamide,
(±)-N-cyclobutyl-6-(7-hydroxy-6,7-dihydro-5H-pyrrolo[1,2-c]imidazol-7-yl)-2-naphthamide,
(±)-6-(7-hydroxy-6,7-dihydro-5H-pyrrolo[1,2-c]imidazol-7-yl)-N-isopropyl-2-naphthamide,
(±)-6-(7-hydroxy-6,7-dihydro-5H-pyrrolo[1,2-c]imidazol-7-yl)-2-naphthamide,
(+)-7-(5-methoxybenzo[b]thiophen-2-yl)-6,7-dihydro-5H-pyrrolo[1,2-c]imidazol-7-ol,
(+)-7-(5-fluorobenzo[b]thiophen-2-yl)-6,7-dihydro-5H-pyrrolo[1,2-c]imidazol-7-ol,
(+)-7-(4′-fluoro[1,1′-biphenyl]-3-yl)-6,7-dihydro-5H-pyrrolo[1,2-c]imidazol-7-ol,
(+)-7-(4′-fluoro[1,1′-biphenyl]-4-yl)-6,7-dihydro-5H-pyrrolo[1,2-c]imidazol-7-ol,
(+)-6-(7-hydroxy-6,7-dihydro-5H-pyrrolo[1,2-c]imidazol-7-yl)-N-methyl-2-naphthamide,
(+)-N-cyclopropyl-6-(7-hydroxy-6,7-dihydro-5H-pyrrolo[1,2-c]imidazol-7-yl)-2-naphthamide,
(+)-N-ethyl-6-(7-hydroxy-6,7-dihydro-5H-pyrrolo[1,2-c]imidazol-7-yl)-2-naphthamide,
(+)-N-cyclobutyl-6-(7-hydroxy-6,7-dihydro-5H-pyrrolo[1,2-c]imidazol-7-yl)-2-naphthamide,
(+)-6-(7-hydroxy-6,7-dihydro-5H-pyrrolo[1,2-c]imidazol-7-yl)-N-isopropyl-2-naphthamide,
(+)-6-(7-hydroxy-6,7-dihydro-5H-pyrrolo[1,2-c]imidazol-7-yl)-2-naphthamide,
(−)-7-(5-methoxybenzo[b]thiophen-2-yl)-6,7-dihydro-5H-pyrrolo[1,2-c]imidazol-7-ol,
(−)-7-(5-fluorobenzo[b]thiophen-2-yl)-6,7-dihydro-5H-pyrrolo[1,2-c]imidazol-7-ol,
(−)-7-(4′-fluoro[1,1′-biphenyl]-3-yl)-6,7-dihydro-5H-pyrrolo[1,2-c]imidazol-7-ol,
(−)-7-(4′-fluoro[1,1′-biphenyl]-4-yl)-6,7-dihydro-5H-pyrrolo[1,2-c]imidazol-7-ol,
(−)-6-(7-hydroxy-6,7-dihydro-5H-pyrrolo[1,2-c]imidazol-7-yl)-N-methyl-2-naphthamide,
(−)-N-cyclopropyl-6-(7-hydroxy-6,7-dihydro-5H-pyrrolo[1,2-c]imidazol-7-yl)-2-naphthamide,
(−)-N-ethyl-6-(7-hydroxy-6,7-dihydro-5H-pyrrolo[1,2-c]imidazol-7-yl)-2-naphthamide,
(−)-N-cyclobutyl-6-(7-hydroxy-6,7-dihydro-5H-pyrrolo[1,2-c]imidazol-7-yl)-2-naphthamide,
(−)-6-(7-hydroxy-6,7-dihydro-5H-pyrrolo[1,2-c]imidazol-7-yl)-N-isopropyl-2-naphthamide, and
(−)-6-(7-hydroxy-6,7-dihydro-5H-pyrrolo[1,2-c]imidazol-7-yl)-2-naphthamide.

Compound (I) can be produced by a known method, for example, the methods described in WO 2002/40484, EP-A-1471056 and the like, or a method according thereto. When compound (I) has an optical isomer, an optical isomer resolved from the racemate is also encompassed in the compound of the present invention. The optical isomer can be obtained as independent products by a synthesis means or a separation means (concentration, solvent extraction, column chromatography, recrystallization and the like) known per se.

Examples of the salt of compound (I) include acid addition salts, such as inorganic acid salts (e.g., hydrochloride, hydrosulfate, hydrobromide, phosphate etc.), organic acid salts (e.g., acetate, trifluoroacetate, succinate, maleate, fumarate, propionate, citrate, tartrate, lactate, oxalate, methanesulfonate, p-toluenesulfonate etc.) and the like. The salt of compound (I) may be a hydrate.

The prodrug of compound (I) refers to a compound which is converted to compound (I) by an in vivo reaction under the action of enzyme, gastric acid or the like.

Examples of the prodrug of compound (I) include compounds resulting from acylation or alkylation of the imidazole nitrogen of compound (I) (e.g., compound which is subjected to dimethylaminosulfonylation, acetoxymethylation, (5-methyl-2-oxo-1,3-dioxolen-4-yl)methoxycarbonylmethylation, pivaloyloxymethylation, benzyloxymethylation, etc.); compounds resulting from acylation, alkylation, phosphorylation, sulfation or boration of the hydroxyl group of compound (I) (e.g., compound (I) in which the hydroxyl group is acetylated, palmitoylated, propanoylated, pivaloylated, succinylated, fumarylated, alanylated, dimethylaminomethylcarbonylated etc.); and the like. These compounds can be prepared by those methods known per se in the art.

The prodrug of compound (I) may exist as such or as a pharmaceutically acceptable salt. Examples of such salt include, in the case where the prodrug of compound (I) has an acidic group such as carboxyl group, salts formed with inorganic bases (e.g., alkali metals such as sodium and potassium; alkaline earth metals such as calcium and magnesium; transition metals such as zinc, iron and copper; etc.), organic bases (e.g., organic amines such as trimethylamine, triethylamine, pyridine, picoline, ethanolamine, diethanolamine, triethanolamine, dicyclohexylamine, N,N′-dibenzylethylenediamine, etc.; so basic amino acids such as arginine, lysine or ornithine; etc.), and the like. When the prodrug of compound (I) has a basic group such as amino group and the like, salts formed with inorganic acid or organic acid (e.g., hydrochloric acid, nitric acid, sulfuric acid, phosphoric acid, carbonic acid, bicarbonic acid, formic acid, acetic acid, propionic acid, trifluoroacetic acid, fumaric acid, oxalic acid, tartaric acid, maleic acid, citric acid, succinic acid, malic acid, methanesulfonic acid, benzenesulfonic acid, p-toluenesulfonic acid etc.), acidic amino acids such as aspartic acid, glutamic acid etc., and the like can be mentioned.

Moreover, the prodrug of compound (I) may be a hydrate or non-hydrate.

A prodrug of compound (I) etc. may be a compound that converts to compound (I) under physiological conditions as described in Development of Pharmaceutical Products, vol. 7, Molecule Design, 163-198, Hirokawa Shoten (1990).

A steroid C_{17,20} lyase inhibitor (particularly, compound (I) or a salt thereof or a prodrug thereof (hereinafter these are collectively referred to as “compound (I′)”)) provides superior effects of suppression of tumor growth in patients with androgen-independent prostate cancer, low toxicity and a fewer side effects. Accordingly, a drug for the prophylaxis or treatment of AIPC of the present invention containing a steroid C_{17,20} lyase inhibitor (particularly, compound (I′)) is useful for mammals (e.g., human, monkey, particularly human).

As compound (I′),
(+)-7-(4′-fluoro[1,1′-biphenyl]-3-yl)-6,7-dihydro-5H-pyrrolo[1,2-c]imidazol-7-ol or a salt thereof,
(−)-7-(4′-fluoro[1,1′-biphenyl]-3-yl)-6,7-dihydro-5H-pyrrolo[1,2-c]imidazol-7-ol or a salt thereof,
(+)-7-(4′-fluoro[1,1′-biphenyl]-4-yl)-6,7-dihydro-5H-pyrrolo[1,2-c]imidazol-7-ol or a salt thereof,
(−)-7-(4′-fluoro[1,1′-biphenyl]-4-yl)-6,7-dihydro-5H-pyrrolo[1,2-c]imidazol-7-ol or a salt thereof,
(+)-6-(7-hydroxy-6,7-dihydro-5H-pyrrolo[1,2-c]imidazol-7-yl)-N-methyl-2-naphthamide or a salt thereof,
(−)-6-(7-hydroxy-6,7-dihydro-5H-pyrrolo[1,2-c]imidazol-7-yl)-N-methyl-2-naphthamide or a salt thereof,
(+)-6-(7-hydroxy-6,7-dihydro-5H-pyrrolo[1,2-c]imidazol-7-yl)-2-naphthamide or a salt thereof,
(−)-6-(7-hydroxy-6,7-dihydro-5H-pyrrolo[1,2-c]imidazol-7-yl)-2-naphthamide or a salt thereof is particularly preferable.

The drug for the prophylaxis or treatment of AIPC of the present invention may be a pharmaceutical agent containing a concomitant drug in combination. By combining a drug for the prophylaxis or treatment of AIPC of the present invention containing compound (I′) as an active ingredient and a concomitant drug, a prophylactic or therapeutic effect on androgen-independent prostate cancer can be enhanced still more.

In the present invention, a concomitant drug is a concept including an anticancer drug.

While the concomitant drug is not particularly limited, one or more kinds selected from, for example, a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof can be used.

Examples of the “sex hormone drug” include fosfestrol, diethylstilbestrol, chlorotrianisene, medroxyprogesterone acetate, megestrol acetate, chlormadinone acetate, cyproterone acetate, danazol, allylestrenol, gestrinone, mepartricin, raloxifene, ormeloxifene, levormeloxifene, anti-estrogen drugs (e.g., tamoxifen citrate, toremifene citrate etc.), pill preparation, mepitiostane, testlactone, aminoglutethimide, GnRH receptor modulators [GnRH receptor agonist (e.g., goserelin acetate, buserelin acetate, leuprorelin acetate etc.), GnRH receptor antagonists (e.g., ganirelix, cetrorelix, abarelix etc.)], droloxifene, epitiostanol, ethinylestradiol sulfonate, aromatase inhibitors (e.g., fadrozole hydrochloride, anastrozole, letrozole, exemestane, vorozole, formestane etc.), anti-androgen drugs (e.g., flutamide, bicalutamide, nilutamide etc.), 5α-reductase inhibitors (e.g., finasteride, epristeride, dutasteride etc.), adrenocorticohormone drugs (e.g., cortisol, dexamethasone, prednisolone, betamethasone, triamcinolone etc.), androgen synthesis inhibitors (e.g., abiraterone etc.), retinoid and drugs that retard retinoid metabolism (e.g., liarozole etc.), ER down-regulators (e.g., fulvestrant (Faslodex (trademark)) etc.) and the like.

Examples of the “alkylating drug” include nitrogen mustard, nitrogen mustard-N-oxide hydrochloride, chlorambutyl, cyclophosphamide, ifosfamide, thiotepa, carboquone, improsulfan tosylate, busulfan, nimustine hydrochloride, mitobronitol, melphalan, dacarbazine, ranimustine, estramustine phosphate sodium, triethylenemelamine, carmustine, lomustine, streptozocin, pipobroman, etoglucid, carboplatin, cisplatin, miboplatin, nedaplatin, oxaliplatin, satraplatin, altretamine, ambamustine, dibrospidium hydrochloride, fotemustine, prednimustine, pumitepa, ribomustin, temozolomide, treosulphan, trophosphamide, zinostatin stimalamer, adozelesin, cystemustine, bizelesin and the like.

Examples of the “antimetabolic drug” include mercaptopurine, 6-mercaptopurine riboside, thioinosine, methotrexate, enocitabine, cytarabine, cytarabine ocfosfate, ancitabine hydrochloride, 5-FU drugs (e.g., fluorouracil, tegafur, UFT, doxifluridine, carmofur, gallocitabine, emitefur etc.), aminopterin, leucovorin calcium, tabloid, butocine, folinate calcium, levofolinate calcium, cladribine, fludarabine, gemcitabine, hydroxycarbamide, pentostatin, piritrexim, idoxuridine, mitoguazone, tiazofurine, ambamustine and the like.

Examples of the “anticancer antibiotics” include actinomycin-D, actinomycin-C, mitomycin C, chromomycin A3, bleomycin hydrochloride, bleomycin sulfate, peplomycin sulfate, daunorubicin hydrochloride, doxorubicin hydrochloride, aclarubicin hydrochloride, pirarubicin hydrochloride, epirubicin hydrochloride, neocarzinostatin, mithramycin, sarcomycin, carzinophilin, mitotane, zorubicin hydrochloride, mitoxantrone hydrochloride, idarubicin hydrochloride and the like.

Examples of the “vegetable alkaloid” include etoposide, etoposide phosphate, vinblastine sulfate, vincristine sulfate, vindesine sulfate, teniposide, paclitaxel, docetaxel, vinorelbine and the like.

Examples of the “immunotherapeutic drug (BRM)” include Picibanil (trademark), Krestin (trademark), sizofuran, lentinan, ubenimex, interferon, interleukin, macrophage colony stimulating factor, granulocyte colony stimulating factor, erythropoietin, lymphotoxin, BCG vaccine, Corynebacterium parvum, levamisole, polysaccharide K, procodazole, cancer vaccine (GVAX (trademark), Sipuleucel-T (Provenge (trademark)), Lapuleucel-T (Neuvenge (trademark)), DCVax-Prostate (trademark), ONCOVEX GM-CSF (trademark), PROSTVAC-VF (trademark), PROMUNE (trademark) etc.) and the like.

As the “cell growth factor” in the “pharmaceutical agents inhibiting the action of cell growth factors or cell growth factor receptors”, any substances that promote cell proliferation, which are normally peptides having a molecular weight of not more than 20,000 that are capable of exhibiting their activity at low concentrations by binding to a receptor, (1) EGF (epidermal growth factor) or substances possessing substantially the same activity as EGF [e.g., EGF, heregulin (HER2 ligand), and the like], (2) insulin or substances possessing substantially the same activity as insulin [e.g., insulin, IGF (insulin-like growth factor)-1, IGF-2, and the like], (3) FGF (fibroblast growth factor) or substances possessing substantially the same activity as FGF [e.g., acidic FGF, basic FGF, KGF (keratinocyte growth factor), FGF-10, and the like], (4) other cell growth factors [e.g., CSF (colony stimulating factor), EPO (erythropoietin), IL-2 (interleukin-2), NGF (nerve growth factor), PDGF (platelet-derived growth factor), TGFβ (transforming growth factor (3), HGF (hepatocyte growth factor), VEGF (vascular endothelial growth factor), etc.], and the like can be mentioned.

Examples of the “cell growth factor receptors” include any receptors capable of binding to the aforementioned cell growth factors, including EGF receptor, HER2 (heregulin receptor), insulin receptor, IGF receptor, FGF receptor-1 or FGF receptor-2, HGF receptor (c-met) and the like.

Examples of the “pharmaceutical agent that inhibits an action of cell growth factor” include HER2 antibody (trastuzumab (Herceptin (trademark)) etc.), EGFR antibody (Cetuximab (Erbitux) (trademark)) etc.), antibody to VEGF (e.g., bevacizumab (Avastin (trademark))), antibody to RANKL (denosumab), antibody to CTLA-4 (ipilimumab), VEGFR antibody, imatinib mesylate, VEGFR inhibitor, EGFR inhibitor (erlotinib (Tarceva (trademark)), tyrosine kinase inhibitors such as gefitinib (Iressa (trademark)) etc.), lapatinib (EGF receptor/HER2 tyrosine kinase inhibitor), sunitinib (tyrosine kinase inhibitor of VEGF receptor-2/PDGF receptor/Kit), sorafenib (kinase inhibitor of Raf kinase/any VEGF receptor), axitinib (tyrosine kinase inhibitor of any VEGF receptor, PDGF receptor β and c-Kit) and the like, ispinesib (kinesin inhibitor), lonafarnib (farnesyl transferase inhibitor), deforolimus (mTOR inhibitor) and ribozyme that suppresses expression of cell growth factor and receptor thereof, antisense drug and the like can be mentioned.

In addition to the above, for example, L-asparaginase, aceglatone, procarbazine hydrochloride, protoporphyrin-cobalt complex salt, mercuric hematoporphyrin-sodium, topoisomerase I inhibitors (e.g., irinotecan, topotecan etc.), topoisomerase II inhibitors (e.g., sobuzoxane etc.), differentiation inducing drugs (e.g., retinoid, vitamin D etc.), angiogenesis inhibitors (e.g., thalidomide, SU11248 etc.), tumor vascular targeting drugs (combretastatin-A-4 prodrug, 5,6-MeXAA), α-blockers (e.g., tamsulosin hydrochloride, naftopidil, urapidil, alfuzosin, terazosin, prazosin, silodosin etc.), serine/threonine kinase inhibitor, endothelin receptor antagonists (e.g., atrasentan, zibotentan etc.), proteasome inhibitors (e.g., bortezomib etc.), Hsp90 inhibitors (e.g., tanespimycin etc.), spironolactone, minoxidil, 11α-hydroxyprogesterone, bone resorption inhibiting/metastasis suppressing agents (e.g., zoledronic acid, alendronic acid, pamidronic acid, etidronic acid, ibandronic acid, clodronic acid) and the like can also be used as concomitant drugs.

As the concomitant drug in the present invention, GnRH receptor modulators [for example, GnRH receptor agonists (e.g., goserelin acetate, buserelin acetate, leuprorelin acetate etc.) or GnRH receptor antagonists (e.g., ganirelix, cetrorelix, abarelix etc.)] are preferable, and GnRH receptor agonists are particularly preferable.

When a drug for the prophylaxis or treatment of AIPC of the present invention is combined with a concomitant drug, the timing of administration of a drug for the prophylaxis or treatment of AIPC and the concomitant drug is not limited. Both concomitant drugs may be simultaneously administered to the subject of administration, or they may be administered in a staggered manner. The drug for the prophylaxis or treatment of AIPC and the concomitant drug may be independently made into preparations, or in the form of a combined agent containing them. The dose of the concomitant drug may be in accordance with a clinically employed dose, which can be appropriately selected according to the subject of administration, administration route, disease, combination and the like. The dose of the concomitant drug is, for example, one-third to 3-fold amount of the dose employed for a concomitant drug as a single agent.

The administration mode of a drug for the prophylaxis or treatment of AIPC of the present invention and a concomitant drug is not particularly limited, and the drug for the prophylaxis or treatment of AIPC and the concomitant drug only need to be combined on administration. Examples of such administration mode include the following:

(1) administration of a single preparation obtained by simultaneously processing the drug for the prophylaxis or treatment of AIPC and a concomitant drug, (2) simultaneous administration of two kinds of preparations of the drug for the prophylaxis or treatment of AIPC and a concomitant drug, which have been separately produced, by the same administration route, (3) administration of two kinds of preparations of the drug for the prophylaxis or treatment of AIPC and a concomitant drug, which have been separately produced, by the same administration route in a staggered manner, (4) simultaneous administration of two kinds of preparations of the drug for the prophylaxis or treatment of AIPC and a concomitant drug, which have been separately produced, by different administration routes, (5) administration of two kinds of preparations of the drug for the prophylaxis or treatment of AIPC and a concomitant drug, which have been separately produced, by different administration routes in a staggered manner (e.g., administration in the order of the drug for the prophylaxis or treatment of AIPC and a concomitant drug, or in the reverse order) and the like.

By combining a drug for the prophylaxis or treatment of AIPC of the present invention and a concomitant drug, the following superior effects can be obtained.

(1) The doses of the drug for the prophylaxis or treatment of AIPC and a concomitant drug can be reduced as compared to a single administration of each of them,
(2) the kind of concomitant drug can be selected according to the symptoms of patients (mild, severe and the like),
(3) by selecting the drug for the prophylaxis or treatment of AIPC and a concomitant drug having different action mechanism, the treatment period can be set long,
(4) by selecting the drug for the prophylaxis or treatment of AIPC and a concomitant drug having different action mechanism, the treatment effect can be prolonged,
(5) a synergistic treatment effect can be obtained by a combined use of the drug for the prophylaxis or treatment of AIPC and a concomitant drug.

When a drug for the prophylaxis or treatment of AIPC of the present invention is administered to patients as a pharmaceutical preparation, a steroid C_{17,20} lyase inhibitor (e.g., compound (I′)) may be formulated into a single preparation, or may be mixed with a concomitant drug, a pharmaceutically acceptable carrier and the like to give a preparation. The proportion of the steroid C_{17,20} lyase inhibitor (e.g., compound (I′)) in a pharmaceutical preparation is generally 0.1-100% (w/w). When a concomitant drug is contained in a pharmaceutical preparation, the proportion of the steroid C_{17,20} lyase inhibitor (e.g., compound (I′)) is generally 0.1-99.9% (w/w).

The dosage form of the above-mentioned pharmaceutical preparation of the present invention for oral administration is, for example, a solid dosage form such as tablet, capsule, granule, powder and the like. The dosage form for parenteral administration such as intravenous, subcutaneous, intramuscular administrations and the like is, for example, injection, suppository, sublingual tablet and the like. The dosage form for sublingual, subcutaneous and intramuscular administrations and the like is, for example, sustained release preparation such as sublingual tablet, microcapsule and the like.

As a pharmaceutically acceptable carrier, for example, various organic or inorganic carrier substances conventionally used as preparation materials are used, which are appropriately blended in suitable amounts with excipient, lubricant, binder, disintegrant and thickener for solid dosage forms; solvent, dispersing agent, dissolution aids, suspending agent, isotonicity agent, buffer and soothing agents for liquid preparations; and the like. Where necessary, additives such as preservative, antioxidant, coloring agent, sweetening agent and the like can also be used according to a conventional method.

Preferable examples of the excipient include lactose, sucrose, D-mannitol, starch, crystalline cellulose, light anhydrous silicic acid and the like. Preferable examples of the lubricant include magnesium stearate, calcium stearate, talc, colloidal silica and the like. Preferable examples of the binder include crystalline cellulose, sucrose, D-mannitol, dextrin, hydroxypropylcellulose, hydroxypropylmethylcellulose, polyvinylpyrrolidone and the like. Preferable examples of the disintegrant include starch, carboxymethylcellulose, calcium carboxymethylcellulose, croscarmellose sodium, carboxymethyl starch sodium and the like. Preferable examples of the thickener include natural rubbers, cellulose derivative, acrylic polymer and the like. Preferable examples of the solvent include water for injection, alcohol, propylene glycol, macrogol, sesame oil, corn oil and the like. Preferable examples of the dispersing agent include Tween 80, HCO 60, polyethylene glycol, carboxymethylcellulose, sodium alginate and the like. Preferable examples of the dissolution aids include polyethylene glycol, propylene glycol, D-mannitol, benzyl benzoate, ethanol, trisaminomethane, cholesterol, triethanolamine, sodium carbonate, sodium citrate and the like. Preferable examples of the suspending agent include surfactants such as stearyltriethanolamine, sodium lauryl sulfate, lauryl aminopropionic acid, lecithin, benzalkonium chloride, benzethonium chloride, glycerol monostearate and the like; hydrophilic polymers such as polyvinyl alcohol, polyvinylpyrrolidone, sodium carboxymethylcellulose, methylcellulose, hydroxymethylcellulose, hydroxyethylcellulose, hydroxypropylcellulose etc.; and the like. Preferable examples of the isotonicity agent include sodium chloride, glycerol, D-mannitol and the like. Preferable examples of the buffer include buffers such as phosphate, acetate, carbonate, citrate etc.; and the like. Preferable examples of the soothing agent include benzyl alcohol and the like. Preferable examples of the preservative include paraoxybenzoates, chlorobutanol, benzyl alcohol, phenethyl alcohol, dehydroacetic acid, sorbic acid and the like. Preferable examples of the antioxidant include sulfite, ascorbic acid and the like.

A pharmaceutical preparation can be produced according to a conventional method. Examples of the production methods are shown below.

### (1) Tablet, Powder, Granule:

They can be produced by adding, for example, excipient, disintegrant, binder, lubricant and the like to a steroid C_{17,20} lyase inhibitor (e.g., compound (I′)), and compression-molding the mixture. For masking of taste or improved enteric property or duration, a coating may be applied after compression-molding.

### (2) Capsule:

It can be produced by filling a steroid C_{17,20} lyase inhibitor (e.g., compound (I′)) in the form of powder, granular or liquid in a capsule, or encapsulation forming with a capsule base. As the starting material of the capsule and capsule base, for example, gelatin, hydroxypropylmethylcellulose and the like can be mentioned.

### (3) Injection:

It can be produced by processing a steroid C_{17,20} lyase inhibitor (e.g., compound (I′)) into an aqueous injection together with, for example, dispersing agent, preservative, isotonicity agent and the like, or by dissolving, suspending or emulsifying a steroid C_{17,20} lyase inhibitor in vegetable oil such as olive oil, sesame oil, cottonseed oil, corn oil and the like, propylene glycol and the like to give an oil injection.

### (4) Suppository:

It can be produced by processing a steroid C_{17,20} lyase inhibitor (e.g., compound (I′)) into an oily or aqueous solid, semisolid or liquid composition. As the oily base to be used for such composition, for example, higher fatty acid glycerides (e.g., cacao butter, Witepsols etc.), intermediate grade fatty acids (e.g., miglyols etc.), vegetable oils (e.g., sesame oil, soybean oil, cottonseed oil etc.) and the like can be mentioned. As the aqueous gel base, for example, natural rubbers, cellulose derivative, vinyl polymer, acrylic polymer and the like can be mentioned.

The administration method of a pharmaceutical preparation can be appropriately selected according to the kind of a steroid C_{17,20} lyase inhibitor (e.g., compound (I′)), the kind of a concomitant drug, the species of the animal selected as the subject of administration and symptoms thereof, dosage form, number of doses and the like. For example, the daily dose of the aforementioned pharmaceutical preparation by oral administration to an adult patient with androgen-independent prostate cancer is generally, about 0.001 to about 500 mg/kg body weight, preferably about 0.1 to about 40 mg/kg body weight, more preferably about 0.5 to about 20 mg/kg body weight, in an effective amount of a steroid C_{17,20} lyase inhibitor (for example, compound (I′)). For parenteral administration and combined use of a steroid C_{17,20} lyase inhibitor (e.g., compound (I′)) and a concomitant drug, the dose is generally smaller than the above-mentioned dose. However, the amount of a steroid C_{17,20} lyase inhibitor (e.g., compound (I′)) to be actually administered is determined according to factors such as the compound selected, various forms of preparation, age, body weight, sex of patients, severity of disease, administration route, administration period and intervals and the like, and can be changed on demand based on the judgment of doctors.

While the administration route of the aforementioned pharmaceutical preparation is not particularly limited, for example, oral or parenteral route can be employed. The “parenteral” in this context includes intravenous, intramuscular, subcutaneous, intranasal, intradermal, instillation, intracerebral, intrarectal, intravaginal and intraperitoneal administrations and the like.

The administration period and intervals of the aforementioned pharmaceutical preparation are changed depending on various situations, and determined on demand based on the judgment of doctors. The administration method includes divided administration, daily administration, intermittent administration, short-term administration of large doses, multiple administration and the like. For example, for oral administration, the dose is desirably administered once or in several portions a day (particularly 2 or 3 portions a day). In addition, a sustained-release preparation may be administered and drip infusion over a long time is also possible.

For the prophylaxis or treatment of androgen-independent prostate cancer, for example, a therapy other than chemical therapy such as operation therapy including orchidectomy, thermotherapy, radiation therapy and the like can also be employed along with a chemical therapy including administration of a drug for the prophylaxis or treatment of AIPC of the present invention.

Moreover, the present invention relates to a therapeutic drug for cancer having resistance to an anticancer drug, which comprises compound (I′) and a concomitant drug in combination.

As compound (I′),
(+)-7-(4′-fluoro[1,1′-biphenyl]-3-yl)-6,7-dihydro-5H-pyrrolo[1,2-c]imidazol-7-ol or a salt thereof,
(−)-7-(4′-fluoro[1,1′-biphenyl]-3-yl)-6,7-dihydro-5H-pyrrolo[1,2-c]imidazol-7-ol or a salt thereof,
(+)-7-(4′-fluoro[1,1′-biphenyl]-4-yl)-6,7-dihydro-5H-pyrrolo[1,2-c]imidazol-7-ol or a salt thereof,
(−)-7-(4′-fluoro[1,1′-biphenyl]-4-yl)-6,7-dihydro-5H-pyrrolo[1,2-c]imidazol-7-ol or a salt thereof,
(+)-6-(7-hydroxy-6,7-dihydro-5H-pyrrolo[1,2-c]imidazol-7-yl)-N-methyl-2-naphthamide or a salt thereof,
(−)-6-(7-hydroxy-6,7-dihydro-5H-pyrrolo[1,2-c]imidazol-7-yl)-N-methyl-2-naphthamide or a salt thereof,
(+)-6-(7-hydroxy-6,7-dihydro-5H-pyrrolo[1,2-c]imidazol-7-yl)-2-naphthamide or a salt thereof, and
(−)-6-(7-hydroxy-6,7-dihydro-5H-pyrrolo[1,2-c]imidazol-7-yl)-2-naphthamide or a salt thereof are particularly preferable.

While anticancer drug is not particularly limited, for example, one or more kinds selected from a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor and a receptor thereof can be mentioned. As the concomitant drug, those similar to the concomitant drugs that can be used concurrently with the aforementioned drug for the prophylaxis or treatment of AIPC can be specifically mentioned. Particularly, as the anticancer drug, GnRH receptor modulators [for example, GnRH receptor agonist (e.g., goserelin acetate, buserelin acetate, leuprorelin acetate etc.), GnRH receptor antagonist (e.g., ganirelix, cetrorelix, abarelix etc.)] can be mentioned.

While cancer is not particularly limited, for example, prostate cancer, androgen-independent prostate cancer, breast cancer, cancer of the uterine body, ovarian cancer, non-small cell lung cancer, urinary bladder cancer, colorectal cancer and esophagus cancer can be mentioned, and prostate cancer and androgen-independent prostate cancer can be particularly mentioned.

The “resistance to an anticancer drug” means that the efficacy is degraded due to repetitive use of an anticancer drug, and the dose needs to be increased to afford the effect obtained when use of the therapeutic drug was started.

The cancer having resistance to an anticancer drug include, for example, cancer wherein tumor recurrence or metastasis due to acquisition of resistance of tumor to a therapeutic drug is observed, cancer for which anticancer drugs are exclusively administered as a treatment, and cancer for which administration of anticancer drugs and other therapy (surgery treatment, radiation therapy, cryotherapy etc.) have been applied. When cancer is prostate cancer or androgen-independent prostate cancer, the “cancer having resistance to an anticancer drug” means a cancer where continuous increase of blood PSA level, tumor growth by a method such as CT, MRI, ultrasonication and the like, expression or aggravation of bone pain, or new metastatic focus is observed after once suppressing the growth ability of tumor by a therapy for inhibiting the production or function of androgen. The continuous increase of blood PSA level means a state where, for example, an increase of the blood PSA level is observed two or more times successively by periodic check ups.

Examples of the concomitant drug to be combined with the therapeutic drug include one or more kinds selected from a sex hormone drug, an alkylating drug, an antimetabolic drug, an anticancer antibiotic, vegetable alkaloid, an immunotherapeutic drug, a molecularly-targeted drug, and a pharmaceutical agent that inhibits the action of a cell growth factor or a receptor thereof. As the concomitant drug, those similar to the concomitant drugs that can be used concurrently with the aforementioned drug for the prophylaxis or treatment of AIPC can be specifically mentioned. As the concomitant drug, GnRH receptor modulators [for example, GnRH receptor agonists (e.g., goserelin acetate, buserelin acetate, leuprorelin acetate etc.) and GnRH receptor antagonists (e.g., ganirelix, cetrorelix, abarelix etc.)] are preferable, and a GnRH receptor agonist is particularly preferable.

The therapeutic drug can be formulated into a preparation by combining compound (I′) and a concomitant drug by a conventional method. Compound (I′) and a concomitant drug, which are the active ingredients, may be independently made into preparations or may be mixed to give a preparation. The dosage form of the pharmaceutical agent for oral administration is, for example, a solid dosage form such as tablet, capsule, granule, powder and the like. The dosage form for parenteral administration such as intravenous, subcutaneous, intramuscular administrations and the like is, for example, injection, suppository, sublingual tablet and the like. The dosage form for sublingual, subcutaneous and intramuscular administrations and the like is, for example, sustained release preparation such as sublingual tablet, microcapsule and the like. As a specific preparation method, those similar to the methods exemplified for the aforementioned drug for the prophylaxis or treatment of AIPC of the present invention or a method in accordance therewith can be used.

The method of administration of the therapeutic drug to a patient can be appropriately selected according to, the kind of compound (I′) to be selected, the kind of the concomitant drug, the species of the animal selected as the subject of administration and symptoms thereof, dosage form, number of doses and the like. As a specific administration method, those similar to the methods exemplified for combination of the aforementioned drug for the prophylaxis or treatment of AIPC of the present invention and a concomitant drug or a method in accordance therewith can be used.

The therapeutic drug is useful for administration to cancer patients who have acquired resistance to an anti-cancer drug, particularly, patients with androgen-independent prostate cancer.

In addition, the present invention relates to a drug for preventing acquisition of resistance of cancer to an anticancer drug, which is a prophylactic drug comprising compound (I′) and a concomitant drug in combination.

As compound (I′),
(+)-7-(4′-fluoro[1,1′-biphenyl]-3-yl)-6,7-dihydro-5H-pyrrolo[1,2-c]imidazol-7-ol or a salt thereof,
(−)-7-(4′-fluoro[1,1′-biphenyl]-3-yl)-6,7-dihydro-5H-pyrrolo[1,2-c]imidazol-7-ol or a salt thereof,
(+)-7-(4′-fluoro[1,1′-biphenyl]-4-yl)-6,7-dihydro-5H-pyrrolo[1,2-c]imidazol-7-ol or a salt thereof,
(−)-7-(4′-fluoro[1,1′-biphenyl]-4-yl)-6,7-dihydro-5H-pyrrolo[1,2-c]imidazol-7-ol or a salt thereof,
(+)-6-(7-hydroxy-6,7-dihydro-5H-pyrrolo[1,2-c]imidazol-7-yl)-N-methyl-2-naphthamide or a salt thereof,
(−)-6-(7-hydroxy-6,7-dihydro-5H-pyrrolo[1,2-c]imidazol-7-yl)-N-methyl-2-naphthamide or a salt thereof,
(+)-6-(7-hydroxy-6,7-dihydro-5H-pyrrolo[1,2-c]imidazol-7-yl)-2-naphthamide or a salt thereof, and
(−)-6-(7-hydroxy-6,7-dihydro-5H-pyrrolo[1,2-c]imidazol-7-yl)-2-naphthamide or a salt thereof are particularly preferable.

As the concomitant drug, anticancer drug and cancer, those described for the above-mentioned “therapeutic drug for cancer having resistance to an anticancer drug” can be mentioned.

The prophylactic drug can be formulated into a preparation by combining compound (I′) and a concomitant drug by a conventional method. Compound (I′) and a concomitant drug, which are the active ingredients, may be independently made into preparations or may be mixed to give a preparation. The dosage form of the prophylactic drug for oral administration is, for example, a solid dosage form such as tablet, capsule, granule, powder and the like. The dosage form for parenteral administration such as intravenous, subcutaneous, intramuscular administrations and the like is, for example, injection, suppository, sublingual tablet and the like. The dosage form for sublingual, subcutaneous and intramuscular administrations and the like is, for example, sustained release preparation such as sublingual tablet, microcapsule and the like. As a specific preparation method, those similar to the methods exemplified for the aforementioned drug for the prophylaxis or treatment of AIPC of the present invention or a method in accordance therewith can be used.

The method of administration of the prophylactic drug to a patient can be appropriately selected according to, the kind of compound (I′) to be selected, the kind of the concomitant drug, the species of the animal selected as the subject of administration and symptoms thereof, dosage form, number of doses and the like. As specific administration methods, those similar to the aforementioned methods employed for combining a drug for the prophylaxis or treatment of AIPC of the present invention and a concomitant drug or a method in accordance therewith can be used.

Since compound (I′) provides superior effects of low toxicity and a fewer side effects, as mentioned above, the therapeutic drug for cancer having resistance to an anticancer drug and the drug for preventing acquisition of resistance of cancer to an anti-cancer drug of the present invention are useful for mammals (e.g., human, monkey, particularly human).

The present invention is explained in detail in the following Experimental Example and Formulation Examples. They are mere embodiments that do not limit the present invention, and may be modified without departing from the scope of the present invention.

### EXAMPLES

### Experimental Example

(+)-6-(7-Hydroxy-6,7-dihydro-5H-pyrrolo[1,2-c]imidazol-7-yl)-N-methyl-2-naphthamide was used as a test compound belonging to compound (I′). For oral administration, the test compound was weighed and placed in a mortar, 0.5% methylcellulose solution was added thereto, and the mixture was sufficiently admixed with a pestle to give a suspension.

6 to 12-year-old castrated male cynomolguses were divided into two groups (7.5 mg/kg/time test compound administration group and 15 mg/kg/time test compound administration group) each including 3 cynomolguses such that blood DHEA concentration would not be impartial. The test compound was repeatedly administered orally twice a day (bid) for one week. Blood samples were collected twice a day from 3 days prior to the administration through the final day of administration. Vehicle (0.5% methylcellulose) was administered about one month after the completion of administration, and blood samples were collected according to the same schedule. Serum DHEA and serum testosterone concentrations were measured by RIA (radioimmunoassay).

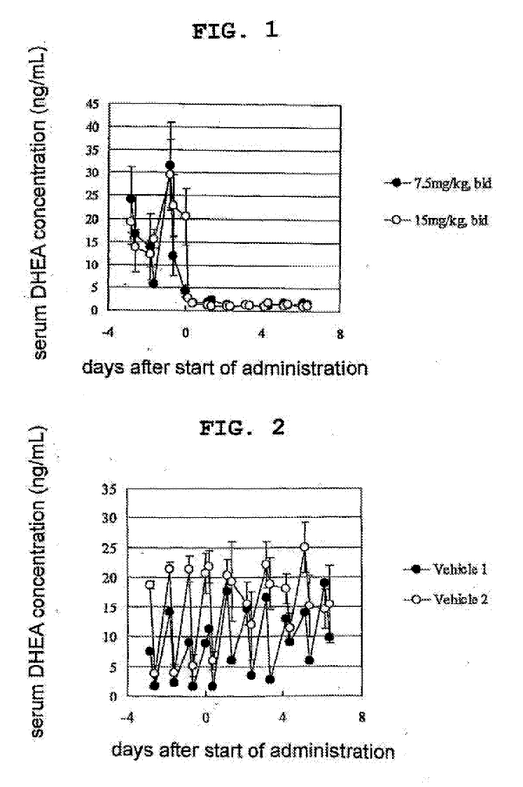

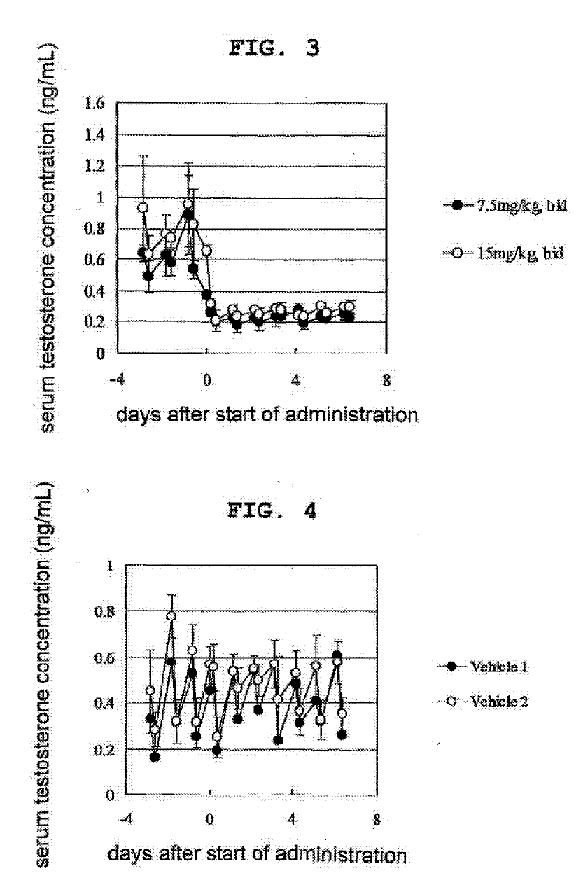

The serum DHEA concentrations of the test compound administration group are shown in FIG. 1, and the serum DHEA concentrations of the vehicle administration group where vehicle was administered about a month after completion of the administration of the test compound are shown in FIG. 2. In addition, the serum testosterone concentrations of the test compound administration group are shown in FIG. 3, and the serum testosterone concentrations of the vehicle administration group where vehicle was administered about a month after completion of the administration of the test compound are shown in FIG. 4.

In androgen-independent prostate cancer patients, the expression of an enzyme which converts less active androgen (for example, DHEA and DHEA-S) produced by the adrenal gland to high-activity androgen (for example, testosterone and dihydrotestosterone) has increased. The serum DHEA concentration and serum testosterone concentration decreased after the start of the administration of the test compound, and the concentrations were maintained at low levels even during the administration period.

It has been shown that compound (I′) is useful as a drug for the prophylaxis or treatment of androgen-independent prostate cancer of the present invention. In addition, it has been shown that compound (I′), when combined with a concomitant drug, is useful as a therapeutic drug for cancer having resistance to an anticancer drug or a drug for preventing acquisition of resistance of cancer to an anticancer drug.

### Formulation Examples

The Formulation Examples of the present invention are described in the following. In the Formulation Examples, for example, one or more kinds selected from (+)-7-(4′-fluoro[1,1′-biphenyl]-3-yl)-6,7-dihydro-5H-pyrrolo[1,2-c]imidazol-7-ol, (−)-7-(4′-fluoro[1,1′-biphenyl]-3-yl)-6,7-dihydro-5H-pyrrolo[1,2-c]imidazol-7-ol, (+)-7-(4′-fluoro[1,1′-biphenyl]-4-yl)-6,7-dihydro-5H-pyrrolo[1,2-c]imidazol-7-ol, (−)-7-(4′-fluoro[1,1′-biphenyl]-4-yl)-6,7-dihydro-5H-pyrrolo[1,2-c]imidazol-7-ol, (+)-6-(7-hydroxy-6,7-dihydro-5H-pyrrolo[1,2-c]imidazol-7-yl)-N-methyl-2-naphthamide, (−)-6-(7-hydroxy-6,7-dihydro-5H-pyrrolo[1,2-c]imidazol-7-yl)-N-methyl-2-naphthamide, (+)-6-(7-hydroxy-6,7-dihydro-5H-pyrrolo[1,2-c]imidazol-7-yl)-2-naphthamide, and (−)-6-(7-hydroxy-6,7-dihydro-5H-pyrrolo[1,2-c]imidazol-7-yl)-2-naphthamide can be used as compound (I).

### Formulation Example 1

| | | |
|---|---|---|
| (1) compound (I) | 1 | g |
| (2) lactose | 197 | g |
| (3) corn starch | 50 | g |
| (4) magnesium stearate | 2 | g |

The above-mentioned (1), (2) and corn starch (20 g) are admixed and granulated together with a paste made from corn starch (15 g) and water (25 mL). Corn starch (15 g) and the above-mentioned (4) are added thereto, and the mixture is compressed by a compression tableting machine to give 2000 tablets having a diameter of 3 mm and containing compound (I) (0.5 mg) per tablet.

### Formulation Example 2

| | | |
|---|---|---|
| (1) compound (I) | 2 | g |
| (2) lactose | 197 | g |
| (3) corn starch | 50 | g |
| (4) magnesium stearate | 2 | g |

In the same manner as in Formulation Example 1, 2000 tablets having a diameter of 3 mm and containing compound (I) (1.0 mg) per tablet are produced.

### Formulation Example 3

| | | |
|---|---|---|
| (1) compound (I) | 5.0 | mg |
| (2) lactose | 60.0 | mg |
| (3) corn starch | 35.0 | mg |
| (4) gelatin | 3.0 | mg |
| (5) magnesium stearate | 2.0 | mg |

A mixture of the above-mentioned (1), (2) and (3) is granulated using a 10% aqueous gelatin solution (0.03 ml, 3.0 mg as gelatin). The granules are passed through 1 mm mesh sieves, dried at 40° C., and sieved again. The obtained granules are mixed with the above-mentioned (5) and compressed. The obtained core tablet is coated with an aqueous sugar coating suspension of saccharose, titanium dioxide, talc and gum arabic. The coated tablet is coated with beeswax to give a coated tablet.

### Formulation Example 4

Titanium oxide (67.5 g) and diiron trioxide (4.05 g) are dispersed in purified water (1575 g). Separately, hydroxypropylmethylcellulose 2910 (manufactured by Shin-Etsu Chemical Co., Ltd. “TC-5” grade R, 502.2 g) and macrogol 6000 (manufactured by Sanyo Chemical Industries, Ltd. “macrogol 6000P”, 101.3 g) are dissolved in purified water (4500 g). They are blended and used as a coating agent.

Compound (I) (2039 g), D-mannitol (2821 g) and crystalline cellulose (manufactured by Asahi Kasei Corporation, “PH101”, 600 g) placed in a fluid bed granulator-dryer (manufactured by POWREX CORPORATION) are premixed in the presence of heated inlet air to give a mixture. An aqueous solution (3000 g) of hydroxypropylcellulose (manufactured by NIPPON SODA CO., LTD. “HPC” grade L, 180 g) is sprayed on the mixture to give a granulated powder. The obtained granulated powder (5076 g) is treated in a power mill (manufactured by Showa Kagaku Kikai Kosakusho Co., Ltd.) to give a milled powder. The obtained milled powder (2256 g), sodium carboxymethyl starch (manufactured by DMV, “Primojel”, 120 g) and magnesium stearate (24 g) are mixed in a tumbler mixer (manufactured by Showa Kagaku Kikai Kosakusho Co., Ltd.) to give a mixed powder. The mixed powder (2220 g) is tableted by a tableting machine (manufactured by KIKUSUI SEISAKUSHO LTD.) to give plain tablets.

A coating agent is sprayed onto the plain tablets in a film coating machine (manufactured by Freund Corporation) to apply 15 mg of coating per tablet, whereby film-coated tablets are obtained.

### INDUSTRIAL APPLICABILITY

The drug for the prophylaxis or treatment of AIPC of the present invention is useful since it can be administered to patients with androgen-independent prostate cancer, posing problems in actual clinical sites. In addition, the therapeutic drug for cancer having resistance to a therapeutic drug (anticancer drug) of the present invention is useful for administration to cancer patients who acquired resistance to an anticancer drug. Moreover, the drug for preventing acquisition of resistance of cancer to an anticancer drug of the present invention is useful since it can be administered to patients for prevention of cancer recurrence.

This application is based on a patent application No. 2007-280813 filed in Japan, the contents of which are incorporated in full herein by this reference.

1. A method for preventing or treating androgen-independent prostate cancer in a mammal, which comprises administering an effective amount of a steroid C_{17,20} lyase inhibitor to said mammal.

2. The method of claim 1, wherein the steroid C_{17,20} lyase inhibitor is a compound represented by the formula (I): 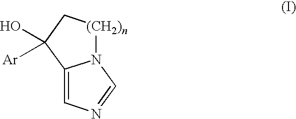
wherein n is an integer of 1 to 3 and Ar is an aromatic ring optionally having substituent(s), or a salt thereof or a prodrug thereof.

3. The method of claim 2, wherein the Ar is a monocyclic or bicyclic aromatic fused ring optionally having substituent(s).

4. The method of claim 2, wherein the Ar is an optionally substituted aromatic ring having, as ring-constituting atom(s), 5 to 10 atoms including 0 to 4 hetero atoms as ring-constituting atom(s), which ring has a bond at a carbon atom.

5. The method of claim 2, wherein the Ar is a group represented by the formula: 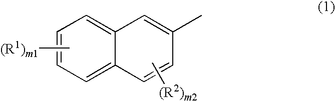
wherein m1 is an integer of 1 to 4, m2 is an integer of 0 to 3, and R¹ and R² are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s), a group represented by the formula: 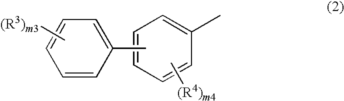
wherein m3 is an integer of 1 to 5, m4 is an integer of 0 to 4, R³ and R⁴ are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s), or a group represented by the formula: 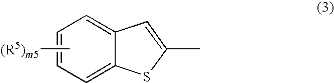
wherein m5 is an integer of 1 to 4, R⁵ is a hydrogen atom, a hydroxyl group optionally having substituent(s), a thiol group optionally having substituent(s), an amino group optionally having substituent(s), an acyl group, a halogen atom or a hydrocarbon group optionally having substituent(s).

6. The method of claim 2, wherein the Ar is a group represented by the formula: 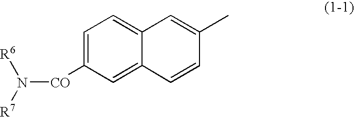
wherein R⁶ and R⁷ are the same or different and each independently is a hydrogen atom or a lower alkyl group, or a group represented by the formula: 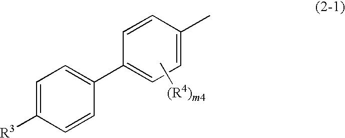
wherein m4 is an integer of 0 to 4, and R³ and R⁴ are the same or different and each independently is a hydrogen atom, a hydroxyl group optionally having substituent(s), a thiol group optionally having substituent(s), an amino group optionally having substituent(s), an acyl group, a halogen atom or a hydrocarbon group optionally having substituent(s).

7. The method of claim 2, wherein the Ar is a group represented by the formula: 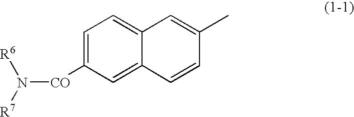
wherein R⁶ and R⁷ are the same or different and each independently is a hydrogen atom or a lower alkyl group.

8. The method of claim 2, wherein the compound represented by the formula (I) is an enantiomer wherein the steric configuration of hydrocarbon bonded to a hydroxyl group is an S configuration.

9. The method of claim 2, wherein the compound represented by the formula (I) is an enantiomer wherein the steric configuration of hydrocarbon bonded to a hydroxyl group is an R configuration.

10. The method of claim 2, wherein the compound represented by the formula (I) is selected from the group consisting of the following compounds:
(±)-7-(5-methoxybenzo[b]thiophen-2-yl)-6,7-dihydro-5H-pyrrolo[1,2-c]imidazol-7-ol,
(±)-7-(5-fluorobenzo[b]thiophen-2-yl)-6,7-dihydro-5H-pyrrolo[1,2-c]imidazol-7-ol,
(±)-7-(4′-fluoro[1,1′-biphenyl]-3-yl)-6,7-dihydro-5H-pyrrolo[1,2-c]imidazol-7-ol,
(±)-7-(4′-fluoro[1,1′-biphenyl]-4-yl)-6,7-dihydro-5H-pyrrolo[1,2-c]imidazol-7-ol,
(±)-6-(7-hydroxy-6,7-dihydro-5H-pyrrolo[1,2-c]imidazol-7-yl)-N-methyl-2-naphthamide,
(±)-N-ethyl-6-(7-hydroxy-6,7-dihydro-5H-pyrrolo[1,2-c]imidazol-7-yl)-2-naphthamide,
(±)-6-(7-hydroxy-6,7-dihydro-5H-pyrrolo[1,2-c]imidazol-7-yl)-N-isopropyl-2-naphthamide,
and (±)-6-(7-hydroxy-6,7-dihydro-5H-pyrrolo[1,2-c]imidazol-7-yl)-2-naphthamide.

11. The method of claim 2, wherein the compound represented by the formula (I) is selected from the group consisting of the following compounds:
(±)-7-(4′-fluoro[1,1′-biphenyl]-3-yl)-6,7-dihydro-5H-pyrrolo[1,2-c]imidazol-7-ol,
(±)-7-(4′-fluoro[1,1′-biphenyl]-4-yl)-6,7-dihydro-5H-pyrrolo[1,2-c]imidazol-7-ol,
(±)-6-(7-hydroxy-6,7-dihydro-5H-pyrrolo[1,2-c]imidazol-7-yl)-N-methyl-2-naphthamide, and
(±)-6-(7-hydroxy-6,7-dihydro-5H-pyrrolo[1,2-c]imidazol-7-yl)-2-naphthamide.

12. A method for preventing or treating androgen-independent prostate cancer in a mammal, which comprises administering an effective amount of (+)-7-(4′-fluoro[1,1′-biphenyl]-3-yl)-6,7-dihydro-5H-pyrrolo[1,2-c]imidazol-7-ol or a salt thereof to said mammal.

13. A method for preventing or treating androgen-independent prostate cancer in a mammal, which comprises administering an effective amount of (−)-7-(4′-fluoro[1,1′-biphenyl]-3-yl)-6,7-dihydro-5H-pyrrolo[1,2-c]imidazol-7-ol or a salt thereof to said mammal.

14. A method for preventing or treating androgen-independent prostate cancer in a mammal, which comprises administering an effective amount of (+)-7-(4′-fluoro[1,1′-biphenyl]-4-yl)-6,7-dihydro-5H-pyrrolo[1,2-c]imidazol-7-ol or a salt thereof to said mammal.

15. A method for preventing or treating of androgen-independent prostate cancer in a mammal, which comprises administering an effective amount of (−)-7-(4′-fluoro[1,1′-biphenyl]-4-yl)-6,7-dihydro-5H-pyrrolo[1,2-c]imidazol-7-ol or a salt thereof to said mammal.

16. A method for preventing or treating of androgen-independent prostate cancer in a mammal, which comprises administering an effective amount of (+)-6-(7-hydroxy-6,7-dihydro-5H-pyrrolo[1,2-c]imidazol-7-yl)-N-methyl-2-naphthamide or a salt thereof to said mammal.

17. A method for preventing or treating androgen-independent prostate cancer in a mammal, which comprises administering an effective amount of (−)-6-(7 hydroxy-6,7-dihydro-5H-pyrrolo[1,2-c]imidazol-7-yl)-N-methyl-2-naphthamide or a salt thereof to said mammal.

18. A method for preventing or treating androgen-independent prostate cancer in a mammal, which comprises administering an effective amount of (+)-6-(7-hydroxy-6,7-dihydro-5H-pyrrolo[1,2-c]imidazol-7-yl)-2-naphthamide or a salt thereof to said mammal.

19. A method for preventing or treating androgen-independent prostate cancer in a mammal, which comprises administering an effective amount of (−)-6-(7-hydroxy-6,7-dihydro-5H-pyrrolo[1,2-c]imidazol-7-yl)-2-naphthamide or a salt thereof to said mammal.

20. The method of claim 1, wherein the steroid C_{17,20} lyase inhibitor is administered in combination with an effective amount of a concomitant drug.

21. The method of claim 20, wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a medicament that inhibits the action of a cell growth factor or a receptor thereof.

22. The method of claim 20, wherein the concomitant drug is a GnRH receptor agonist or a GnRH receptor antagonist.

23. A method for treating mammalian cancer having resistance to an anticancer drug, which comprises administering an effective amount of a compound represented by the formula (I): 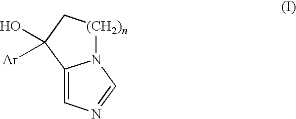
wherein n is an integer of 1 to 3 and Ar is an aromatic ring optionally having substituent(s), or a salt thereof or a prodrug thereof, and an effective amount of a concomitant drug in combination.

24. The method of claim 23, wherein the anticancer drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a medicament that inhibits the action of a cell growth factor or a receptor thereof.

25. The method of claim 23, wherein the anticancer drug is a GnRH receptor agonist or a GnRH receptor antagonist.

26. The method of claim 23, wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a medicament that inhibits the action of a cell growth factor or a receptor thereof.

27. The method of claim 23, wherein the concomitant drug is a GnRH receptor agonist or a GnRH receptor antagonist.

28. A method for preventing cancer in a mammal from acquiring resistance to an anticancer drug, which comprises administering an effective amount of a compound represented by the formula (I): 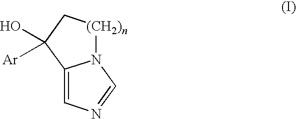
wherein n is an integer of 1 to 3 and Ar is an aromatic ring optionally having substituent(s), or a salt thereof or a prodrug thereof, and an effective amount of a concomitant drug in combination.

29. The method of claim 28, wherein the anticancer drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a medicament that inhibits the action of a cell growth factor or a receptor thereof.

30. The method of claim 28, wherein the anticancer drug is a GnRH receptor agonist or a GnRH receptor antagonist.

31. The method of claim 28, wherein the concomitant drug is one or more kinds selected from the group consisting of a sex hormone drug, an alkylating drug, an antimetabolic drug, an anticancer antibiotic, vegetable alkaloid, an immunotherapeutic drug, a molecularly-targeted drug, and a medicament that inhibits the action of a cell growth factor or a receptor thereof.

32. The method of claim 28, wherein the concomitant drug is a GnRH receptor agonist or a GnRH receptor antagonist.

33-36. (canceled)

37. The method of claim 20, wherein the concomitant drug is prednisolone.

38. The method of claim 23, wherein the concomitant drug is prednisolone.

39. The method of claim 28, wherein the concomitant drug is prednisolone.
